(19) United States
(12) Patent Application Publication
(10) Pub. No.: US 2008/0004528 A1
(43) Pub. Date: Jan. 3, 2008

(54) ULTRASOUND MEDICAL SYSTEM AND METHODS

(76) Inventors: Thomas P. Fitzsimons, Swarthmore, PA (US); Lisa K. Jordan, Boston, MA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/627,232

(22) Filed: Jan. 25, 2007

Related U.S. Application Data
(60) Provisional application No. 60/762,154, filed on Jan. 25, 2006.

(51) Int. Cl. A61B 8/00

(52) U.S. Cl. 600/439; 600/437

(57) ABSTRACT

Provided are ultrasound medical devices for the imaging, characterization, diagnosis, and treatment of patient tissue in or near which an air/tissue interface exists. Also provided are methods for the use of the disclosed ultrasound devices.

### CROSS REFERENCE TO RELATED APPLICATIONS

This application claims benefit of U.S. Provisional Application No. 60/762,154, filed Jan. 25, 2006, the disclosure of which is hereby incorporated by reference in its entirety.

### FIELD OF THE INVENTION

The present invention relates generally to ultrasound, and more particularly to ultrasound medical devices for the imaging, characterization, diagnosis, and treatment of patient tissue in or near which an air/tissue interface exists. Also provided are methods for the use of the disclosed ultrasound devices.

### BACKGROUND OF THE INVENTION

Ultrasound medical systems and methods that are known in the art include the use of ultrasound imaging to identify patient tissue for medical treatment, as well as the use of ultrasound to medically destroy identified patient tissue through the ultrasonic heating thereof. Diagnostic imaging is performed at lower ultrasonic power, and typically utilizes a frequency range of 1-20 MHz, and medical treatment is done at higher power. Kennedy J E et al., Br J Radiol. 76(909):590-9 (2003). Review. Although low power, imaging ultrasound will not medically affect patient tissue, high power medical-treatment ultrasound (known as HIFU—high intensity focused ultrasound), when focused at a focal zone a distance away from the ultrasound source, will substantially medically affect patient tissue in the focal zone. Illing R O et al., Br. J. Cancer. 93(8):890-5 (2005). However, focused medical-treatment ultrasound will not substantially medically affect patient tissue outside the focal zone, including patient tissue located between the source and the focal zone. Kennedy J E et al., Br J Radiol. 76(909):590-9 (2003). Review.

A transducer assembly may include a single or multiple ultrasound transducer(s) having a single transducer element, or an array of transducer elements acting together, to ultrasonically image the patient and to ultrasonically ablate identified patient tissue. It is also known in the art to convert ultrasound imaging data into temperature imaging data for ultrasound-treated patient tissue to monitor the ultrasound treatment. A transducer element may possess a concave shape or an acoustic lens to focus ultrasound energy, and multiple element transducers may include a planar, concave, or convex array of transducer elements to focus ultrasound energy. Kremkau F W, Radiographics. 13(5):1163-76 (1993). Arrays of transducer elements may also include an array whose transducer elements are electronically or mechanically controlled together to steer and focus the ultrasound emitted by the array to a precise focal zone to provide three-dimensional medical ultrasound treatment of patient tissue. Id.

Known ultrasound imaging includes A-mode, B-mode (“brightness”), M-mode, 2D-real time, and various Doppler modes, and may be used for the detection of blood flow, and hence to determine areas of increased vascularization, which may in turn indicate neoplastic activity. See, e.g., Brittenden J et al., Br J Radiol. 68(816):1344-8 (1995). In general, however, ultrasonic imaging is not used within air-filled viscera due to the poor acoustic impedance match between human tissue and air.

Ligand-targeted ultrasonic contrast agents are recognized as having the potential to improve diagnosis by enhancing, for example, the acoustic brightness of the image surface, and to improve treatment of disease by permitting targeted local delivery of therapy. de Lima, J J, Radiat Prot Dosimetry. 115(1-4):51-7 (2005). Clinicians therefore have the option of employing ultrasound imaging with or without the concomitant use of ultrasonic contrast agents.

Recently, screening for early detection of patients at risk for lung cancer and other pathologies has become an important medical practice. Patients are given a CT scan to detect small pre-cancerous nodules or lesions in the lung. Jett J R, Semin Respir Crit Care Med. 21(5):385-92 (2000). Lesions as small as 1 mm³ can be detected in this manner. Current practice calls for such lesions to be biopsied to determine if they are cancerous, and cancerous and dysplastic lesions are generally removed by open surgery. Lesions that are benign are medically tracked to permit the assessment of any changes over time. Both the cost and radiation dose associated with tracking these lesions is significant.

Traditionally, it has been believed that HIFU could not be directed through air-filled viscera, or through other obstructions such as bone, that can absorb or reflect an ultrasound beam. Kennedy J E et al., Br J Radiol. 76(909):590-9 (2003). Review. Accordingly, ultrasound medical treatment of such organs as the lung, colon, or stomach has conventionally been avoided. With respect to the lung, this is due to the fact that the alveoli of the lung contain air that reflects back most of the ultrasound, thereby preventing the ultrasound from effectively penetrating the lung to the location of any lesion. The use of higher power ultrasound for effective penetration of the lung to reach the lesion would injure or destroy the alveoli which are needed for breathing. The use of ultrasound devices in the lung is also limited by the fact that effective ultrasound imaging and treatment requires the existence of a focal zone that is stationary relative to the source of the ultrasound energy, and normal pulmonary motion interferes with the realization of a stable target area.

Thus, although some early detection measures are presently available, what is needed is improved ultrasound medical systems and improved ultrasound medical devices that can be used to image these lesions, guide the biopsy of the lesions or otherwise enable their characterization, and then, if desired, use high intensity focused ultrasound to ablate these lesions. Subsequent scanning may be obviated and enhanced therapeutic utility demonstrated.

### SUMMARY OF THE INVENTION

The present invention relates particularly to ultrasound medical devices for the imaging, characterization, diagnosis, and treatment of patient tissue in or near which an air/tissue interface exists. Also provided are methods for the use of the disclosed ultrasound devices. The present invention permits the in vivo ultrasound tissue characterization and tissue ablation of pathogenic and abnormal tissue near or within air-filled viscera.

In accordance with one embodiment of the present invention, there is provided an ultrasound medical device suitable for use in a target area in or near which an air/tissue interface exists comprising an insertion piece having a longitudinal axis and a distal end insertable into a patient, and, a transducer assembly disposed at the distal end of the insertion piece comprising one or more transducer elements each having an ultrasound emitting surface. The transducer assembly can be adjustably mounted to provide a source of ultrasound that may be adaptively focused onto a desired target area. The transducers may otherwise or also be electronically focusable onto a desired target area. In contrast to what has traditionally been possible through the use of ultrasound devices known in the art, the target area can be in or near the oral cavity or pharynx, nasal cavity or pharynx, esophagus, trachea, bronchi, lungs, stomach, or colon.

The insertion piece of the ultrasound medical device may have a length of about 20 to about 600 cm and has a diameter of about 1 mm to about 30 mm. In some of the instant devices, the insertion piece is a catheter and has a diameter of about 5 mm or smaller.

The insertion piece may be adapted for insertion into a patient through the working channel of a flexible bronchoscope, a rigid bronchoscope, or through the interstitial spaces at the surface of a lung. The insertion piece may be included within or incorporate a bronchoscope. Additionally, the insertion piece may further comprise at least one lumen that is compatible for use with a guide wire. It is also possible for the ultrasound medical device to be adapted for transbronchial or percutaneous insertion into the patient.

In some versions of the instant invention, the one or more transducer elements of the ultrasound medical device are disposed at the terminus of the distal end of the insertion piece. Alternatively, at least one of the one or more transducer elements may be disposed on the side of the distal end of the insertion piece. In other manifestations, at least one of the one or more transducer elements is disposed on the side of the distal end of the insertion piece, and at least one of the transducer elements is also disposed at the terminus of the distal end of the insertion piece.

At least one of the transducer elements may be an ultrasound imaging transducer, and the ultrasound imaging transducer may be capable of A, B M, 2D, Doppler, Pulsed-wave Doppler, Continuous-wave Doppler, Color Doppler, Power Doppler, Directional Power Doppler, or Duplex ultrasound imaging, or any combination thereof. Otherwise, at least one of the one or more transducer elements may be an ultrasound medical treatment transducer. Other devices can be configured so that at least one of the transducer elements is an ultrasound imaging transducer and an ultrasound medical treatment transducer.

The ultrasound emitting surface of at least one of the one or more transducers can be oriented so that a cross-section thereof is at an angle that is substantially perpendicular to the longitudinal axis of the insertion piece. Also possible are configurations wherein the transducer assembly includes a plurality N of transducers and wherein the ultrasound emitting surface of each transducer is oriented at an angle of about 360/N degrees apart from the ultrasound emitting surface of an adjacent transducer. Also provided are configurations in which transducer elements are oriented at irregular intervals around the distal end of the insertion piece, or where the transducer assembly comprises a single transducer placed partially or completely circumferentially around the longitudinal axis of the insertion piece. The transducer assembly may be actively or passively cooled to dissipate heat.

The instant invention also includes an ultrasound medical device wherein at least one of the one or more transducers features an ultrasound emitting surface the cross-section of which is substantially linear. It may also be the case that at least one of the one or more transducers features an ultrasound emitting surface the cross-section of which is substantially concave.

There are also provided ultrasound medical devices suitable for use in a target area in or near which an air/tissue interface exists comprising least one expandable portion disposed on a section or plurality of sections of the distal portion of the insertion piece. The at least one expandable portion may surround at least one of the one or more transducer elements, and may be expanded to contact the surface of said patient's tissue to provide acoustic coupling between the at least one of the one or more transducer elements and the patient tissue. The at least one expandable portion may comprise at least one balloon for anchoring or stabilizing the insertion piece within the patient, or may comprise one or more stents, struts, tines (fixed or retractable), or screws or grasping means (for active fixation) for stabilizing the insertion piece within the patient.

The transducer assembly may be adjustably mounted on a tractable surface having an adaptively variable surface profile that may be actuated electronically, hydraulically, pneumatically, magnetically, chemically, with a shape memory metal, mechanically, or a combination thereof. In some versions, the adaptively variable surface profile varies according to its radius or radii of curvature.

The tractable surface may be a flexible material. The flexible material can comprise a magnetic material that may be deflected magnetically. Other versions are configured so that the tractable surface is a flexible material and the flexible material is deflected by mechanical expansion, compression, or a combination thereof. The flexible material may optionally be in communication with an interior portion of the insertion piece, and in some of these embodiments, flexible material is deflected by a variable vacuum created through the induction of negative pneumatic or hydraulic pressure within the insertion piece, or is deflected through the induction of positive pneumatic or hydraulic pressure within the insertion piece.

The tractable surface may otherwise comprise a plurality of piezoelectric elements, or, the tractable surface can comprise a plurality of plates that can be adjusted by one or more cables.

The ultrasound medical devices may further include a hand-piece operatively connected to the insertion piece. The device can also include an ultrasound controlling unit operatively connected to the insertion piece.

In another aspect, the instant invention is directed to methods of imaging, characterization, diagnosis, and/or medical treatment of patient tissue comprising the steps of introducing into a patient at a location having or suspected of having an air/tissue interface an insertion piece having a longitudinal axis and a distal end, the insertion piece having a transducer assembly disposed at the distal end of said insertion piece, the transducer assembly comprising one or more transducer elements each having an ultrasound emitting surface; guiding the device within the patient to a site of interest; identifying a feature on or in the site of interest for medical treatment; positioning the transducer assembly of said ultrasound medical device at, in, or near the feature; activating the device so that at least part of the transducer assembly thereof begins emitting ultrasound, and, using the ultrasound emissions for imaging or medical treatment, or for any combination thereof.

In some of the disclosed methods, the transducer assembly of the insertion piece is adjustably mounted to provide a source of ultrasound that may be adaptively focused onto a desired target area. The transducer assembly may also or otherwise be electronically focusable onto a desired target area. The insertion piece can further comprise at least one expandable portion disposed on a section or plurality of sections of the distal portion of the insertion piece, wherein the at least one expandable portion surrounds at least one of the one or more transducer elements, and may be expanded to contact the surface of said patient's tissue to provide acoustic coupling between the at least one of the one or more transducer elements and the patient tissue. All combinations of these and the other disclosed features are contemplated as being within the scope of the instant methods.

As contrasted with techniques known in the art, the site of interest may be in or near the oral cavity or pharynx, nasal cavity or pharynx, esophagus, trachea, bronchi, lungs, stomach, or colon.

The insertion piece may be introduced into a patient endoscopically, transbronchially, percutaneously, or may be used during an open surgical procedure. To this end or to others, the insertion piece may comprise at least one lumen that is compatible for use with a guide wire, and in some cases, the insertion piece is introduced into a patient by translation over a guide wire.

The current methods may further comprise adjusting the transducer assembly to focus the ultrasound onto the desired target area.

The imaging or medical treatment step may be repeated one or more times. The instant methods may also include adjusting the transducer assembly to focus the ultrasound onto the desired target area prior to one or more of the repetition or repetitions of the imaging or medical treatment step. The repetition or repetitions of the imaging or medical treatment step may include alternating between imaging and medical treatment, and in yet other embodiments, the alternation between imaging and medical treatment comprises a therapy regimen including one or more episodes of imaging, optionally followed by adjustment of the transducer assembly to focus the ultrasound onto a desired target area, optionally followed by one or more episodes of imaging, followed by one or more episodes of medical treatment. The described therapy regimen may be performed a single time, or may be repeated one or more times, and in some cases, one or more of the repetitions of the therapy regimen may involve selecting a different target area.

Additionally, the imaging and medical treatment may be performed simultaneously.

In some cases, the imaging or medical treatment step comprises imaging, and the imaging comprises diagnosis and/or characterization of the target area. The imaging can comprise assessing the target area for its status as benign, malignant, or unknown; in other embodiments, the imaging can also or otherwise include determining the size, geometry, vascularity, and/or density of the target area. The determination of size, geometry, vascularity, and/or density of the target area can be used to guide the adjustment of the focus of the ultrasound emissions in order to align the focus with a desired location on or in the target area, or it can be used to guide the adjustment of the power of the ultrasound emissions so that the power level is optimally compatible with additional imaging and/or medical treatment of the target area. With respect to the latter case, the disclosed methods can further comprise one or more additional repetitions of imaging or medical treatment or a combination thereof.

The imaging or medical treatment step in some cases comprises medical treatment, and such medical treatment can be applied to the target area in order to reduce the size of the target area; ablate all or part of the target area; induce necrosis in all or part of the target area; induce apoptosis in all or part of the target area; damage or destroy the vasculature associated with the target area; slow or retard the growth of the target area; or, slow or retard the metastasis of the target area; or any combination thereof. In addition, the treatment step may comprise the ultrasonic activation of a locally-delivered pharmacologic agent or the sonoporation of a pharmacologic agent in to the target area.

The imaging or medical treatment step can comprise using A, B M, 2D, Doppler, Pulsed-wave Doppler, Continuous-wave Doppler, Color Doppler, Power Doppler, Directional Power Doppler, or Duplex ultrasound, or any combination thereof.

The provided methods may additionally comprise at least partially expanding at least one of the at least one expandable portion before the imaging or medical treatment step. The at least one expandable portion may be used to anchor or stabilize the insertion piece within the patient.

The instant methods can further include providing an ultrasound contrast agent and delivering the ultrasound contrast agent to the target area.

Further objects and advantages afforded by the invention are apparent from the detailed description provided below.

### BRIEF DESCRIPTION OF THE DRAWINGS

FIG. 1 is an enlarged view of an exemplary insertion piece of the instant ultrasound medical device.

FIGS. 2A to 2E provide exemplary configurations of the ultrasound transducers as they are disposed on the insertion piece's distal end.

FIGS. 3A to 3E depict an embodiment wherein the transducer assembly is adjustably mounted on a tractable surface.

FIG. 4 shows an embodiment that employs a plurality of piezoelectric elements in order to vary the surface on which the transducer assembly is mounted.

FIG. 5 depicts an embodiment of the instant ultrasound medical device that includes an expandable portion.

FIG. 6 depicts the cross-section of an embodiment which incorporates separate lumens for accommodating an optical imaging component, an ultrasound imaging and ablation portion, and channels for cooling of the ablation transducer.

### DETAILED DESCRIPTION OF ILLUSTRATIVE EMBODIMENTS

The present invention is directed to ultrasound medical devices for the imaging, characterization, diagnosis, and treatment of patient tissue in or near which an air/tissue interface exists. Also provided are methods for the use of the disclosed ultrasound devices. The present invention permits the in vivo ultrasound tissue characterization and tissue ablation of pathogenic and damaged tissue near or within air-filled viscera.

It should be noted that the invention is not limited in its form, application, or use to the configurations, arrangements of parts, and steps illustrated in the accompanying drawings and description. The terms and phrases used herein to describe the illustrative embodiments of the instant invention and are for clarification purposes only; they are not intended for the purpose of limiting the scope of the present invention.

The instant ultrasound medical devices provide a functionality that has heretofore been unavailable, namely, the ability to permit ultrasonic imaging and medical treatment of patient tissue located in or near which an air/tissue interface exists. Despite the conventional wisdom that because ultrasound cannot propagate through air-filled viscera such as the lung or bowel, that features such as pulmonary pre-cancerous or cancerous lesions cannot be viable targets for ultrasonic imaging or ablation, the present invention is specifically adapted to permit its effective use in tissue regions where air spaces exist.

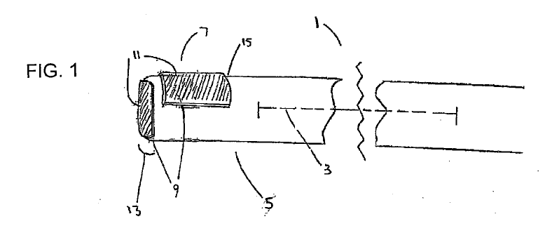

An exemplary embodiment of the present ultrasound medical device is provided in FIG. 1, wherein, as explained below, it may be observed that while the instant devices may be equipped with ultrasound-emitting transducers of a variety that are traditionally known in the art, they are otherwise adapted in a previously unknown and highly useful fashion for their specialized use in or near air-filled viscera. Target areas for the ultrasonic imaging and/or medical treatment as afforded by the instant devices may include those in or near the oral cavity or pharynx, nasal cavity or pharynx, esophagus, trachea, bronchi, lungs, stomach, colon, or other regions where air/tissue interfaces are commonly found, in addition to organs or other tissue that are more traditional targets for ultrasonic imaging and/or high intensity focused ultrasound (“HIFU”) medical treatment.

Referring now to FIG. 1, an exemplary ultrasound medical device includes an insertion piece 1 having a longitudinal axis 3 and a distal end 5 insertable into a patient. Insertion piece 1 may have a length of about 20 to about 600 cm and a diameter of about 1 mm to about 30 mm. In other embodiments, the insertion piece 1 is a catheter and has a diameter of about 5 mm or smaller and is inserted through the working channel of a flexible bronchoscope. The insertion piece 1 may also be adapted for insertion into a patient through a rigid bronchoscope, that is, the insertion piece 1 is of larger diameter than a traditional catheter. The insertion piece 1 can also be adapted for insertion through the interstitial spaces at the surface of a lung. The insertion piece 1 may be constructed from any material that is medically suitable for insertion into a patient, such materials being known in the art, including, for example, silicon, various plastics and polymer materials, and the like. Highly flexible materials are preferred when the insertion piece 1 is a catheter, while greater levels of rigidity are acceptable for insertion pieces that are suitable for use with a bronchoscope. The device may also or otherwise comprise areas of varying flexibility to achieve desired handling characteristics. The proximal end of the insertion piece may have a hand-piece (not pictured) or other control mechanism to remotely manipulate or deflect the distal end 5. The selection of appropriate materials given the desired use of the insertion piece 1 are within the knowledge of those skilled in the art, and may include, inter alia, metals (such as stainless steel, titanium, nitanol, or others), elgiloy, silicon (including silicon and/or silastic), plastics (such as polyurethane, polyethylene, PVC, or others), epoxy, or a combination thereof.

Disposed at the distal end 5 of said insertion piece is a transducer assembly 7 including one or more transducer element(s) 9, each having an ultrasound emitting surface 11. At least one of the one or more transducer elements 9 may be disposed at the terminus 13 of the distal end of the insertion piece 5. In other versions, at least one of the one or more transducer elements 9 is disposed on the side 15 of the distal end of the insertion piece 5, and the device may also be configured so that least one transducer element 9 is disposed on the side 15 of the distal end of the insertion piece 5 and at least one transducer element 9 is disposed at the terminus 13 of the distal end of the insertion piece 5. These various arrangements of the transducer element(s) 9 as disposed at the distal end of the insertion piece 5 permit the application of ultrasound either to a target forward from the terminus 13 of the insertion piece or to the side 15 thereof, or both, as clinically required.

The transducer elements 9 with which the instant device are equipped are constructed with materials well known among skilled artisans. Traditional ultrasonic transducers are constructed from piezoelectric elements, such as, inter alia, multi-layered piezoelectric ceramics, although all appropriate materials and combinations thereof are contemplated. In some preferred embodiments, at least one of the one or more transducer elements is an ultrasound imaging transducer. Imaging ultrasound transducers preferably are capable of A, B M, 2D, Doppler, Pulsed-wave Doppler, Continuous-wave Doppler, Color Doppler, Power Doppler, Directional Power Doppler, or Duplex ultrasound imaging, or any combination thereof, although all modes of ultrasound emission are contemplated as being within the scope of the present invention. Traditionally, diagnostic ultrasound is performed using ultrasound frequencies from about 1-30 MHz, and it is contemplated that the instant imaging ultrasound transducers are capable of producing ultrasound within such a range, inclusive. Additionally, ultrasonic imaging traditionally generates tissue power levels below 100 milliwatts per cubic centimeter, and the instant transducers can be configured to produce power at such levels.

At least one of the one or more transducer elements 9 can be an ultrasound medical treatment transducer. Preferably, medical treatment transducers are capable of producing high intensity focused ultrasound at appropriate frequencies to reduce the size of a feature of patient tissue, ablate all or part of the feature; induce necrosis in all or part of the feature, induce apoptosis in all or part of the feature, damage or destroy the vasculature associated with the feature, slow or retard the growth of the feature, or, slow or retard the metastasis of the feature, or any combination thereof. Although the ultrasound power level that is necessary to effect such results can vary according to the nature of the target area, transducer elements capable of producing power densities above ten watts per cubic centimeter are preferred. Otherwise, the transducer element should be capable of producing temperatures that are sufficiently high to produce the described effects, for example, at or above about 50° C.

The at least one of the one or more transducer elements 9 can be both an ultrasound imaging transducer and an ultrasound medical treatment transducer. Stated differently, such transducer elements are preferably capable of producing ultrasound across the entire range of power densities and frequencies necessary to produce ultrasound for imaging and, separately, medical treatment.

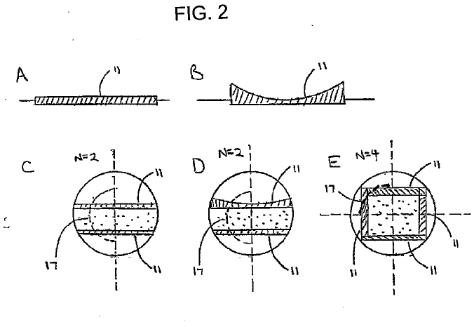

The ultrasound transducer(s) 9 of the instant ultrasound medical device may be present in any desired number and in any appropriate configuration. For example, in some embodiments, the ultrasound emitting surface 11 of at least one of the one or more ultrasound transducers 9 is oriented so that a cross section thereof is at an angle that is substantially perpendicular to the longitudinal axis 3 of the insertion piece 1. FIGS. 2A to 2E provide exemplary configurations of the ultrasound transducer(s) 9 as they are disposed on the insertion piece's distal end 5, whether at the terminus 13 or side 15 thereof, or both. As depicted in FIG. 2A, at least one of the one or more ultrasound transducers 9 may feature an ultrasound emitting surface 11 the cross-section of which is substantially linear. By contrast, FIG. 2B provides an exemplary ultrasound transducer 9 having an ultrasound emitting surface 11 the cross-section of which is substantially concave. With respect to embodiments in which the ultrasound transducer 9 has a substantially concave ultrasound emitting surface 11, the concavity of the ultrasound emitting surface may be vary as is preferred, where such variations may be incorporated in order to vary the focal zone of the ultrasound emitted therefrom.

The transducer assembly 7 of the ultrasound medical device may include a plurality N of transducers 9, wherein the ultrasound emitting surface 11 of each transducer is oriented substantially at an angle 17 of about 360/N degrees apart from the ultrasound emitting surface of an adjacent transducer. In FIG. 2C, N equals 2, and the respective ultrasound emitting surfaces 11 of each transducer are oriented substantially at an angle 17 of 180° apart from the ultrasound emitting surface of the adjacent transducer. In FIG. 2D, N again equals 2, and the angle of separation between the respective ultrasound emitting surfaces 11 of the transducers is still substantially 180°, although the cross-section of the ultrasound emitting surface 9 of the transducer that is oriented towards the top of the figure is substantially concave; FIG. 2D is therefore intended to demonstrate that all combinations of transducers having linear and/or concave ultrasound emitting surfaces are contemplated. In FIG. 2E, N equals 4, and the respective ultrasound emitting surfaces 11 of each transducer are oriented substantially at an angle 17 of 90° apart from the ultrasound emitting surface of the adjacent transducer. Any number N of transducer elements and any combination of transducer elements with substantially planar and substantially concave ultrasound emitting surfaces may be used and are considered to be within the scope of the present invention. Also contemplated are configurations in which transducer elements are oriented at irregular intervals around the distal end of the insertion piece 1, or where the transducer assembly comprises a single, round element placed circumferentially around the longitudinal axis 3 of the insertion piece.

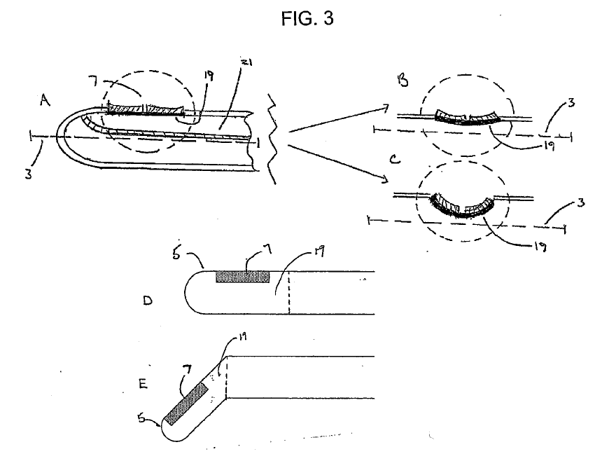

Part or all of the transducer assembly 7 disposed at the distal end 5 of the insertion piece may be adjustably mounted to provide a source of ultrasound that may be adaptively focused onto a desired target area. For example, as depicted in FIG. 3A, in some embodiments, part or all the transducer assembly 7 is adjustably mounted on a tractable surface 19 having an adaptively variable surface profile that may be actuated electronically, hydraulically, pneumatically, magnetically, chemically, through a shape memory metal, mechanically, or a combination thereof. In such instances, the adaptively variable surface profile may vary according to its radius or radii of curvature. In FIG. 3A, for example, the radius of curvature of the surface profile of the tractable surface 19 is substantially aligned with the longitudinal axis of the insertion piece 3, which in this context is depicted for reference purposes only. However, in FIGS. 3B and 3C, it can be observed that the radius of curvature of the surface profile of the tractable surface 19 may be deflected relative to the longitudinal axis of the insertion piece 3. FIGS. 3D and 3E provide another embodiment of the instant invention in which the transducer assembly 7 is adjustably mounted on a tractable surface 19 having an adaptively variable surface profile.

The tractable surface 19 may be a flexible material. The flexible material may in turn be a magnetic material that may be deflected magnetically, as controlled by the operator of the device. In other versions of the instant invention, the flexible material is deflected by mechanical expansion, compression, or a combination thereof. The tractable surface 19 may optionally be in communication with an interior portion 21 of the insertion piece, as depicted in FIG. 3A. In some embodiments wherein the tractable surface 19 is a flexible material and is in communication with an interior portion 21, the flexible material may be deflected by a variable vacuum created through the induction of negative pneumatic or hydraulic pressure within the interior portion 21, or through the induction of positive pneumatic or hydraulic pressure within the interior portion 21.

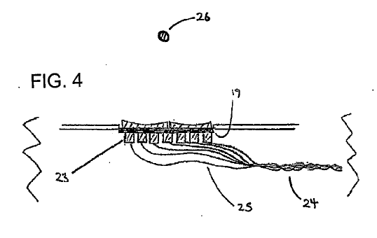

As shown in FIG. 4, the tractable surface 19 can comprise a plurality of piezoelectric elements 23; in such embodiments, the tractable surface may be deflected through the induction of an appropriate voltage transmitted via electrically conducting materials 25 which may be contained in a cable bundle 24 affecting some or all of the piezoelectric elements 23. The piezoelectric elements can be electrically pulsed simultaneously, or with slight time delays to electronically steer the emitted ultrasound, focusing it at a targeted point in space 26. The transducer elements making up a transducer assembly need not be mounted upon a tractable surface in order to affect variable focusing of the emitted ultrasound. The mechanics of movement of distal portions of insertable medical devices are well known in the art and are described for example in U.S. Pat. No. 6,123,699. The ultrasound may additionally or alternatively be emitted from transducer elements that are collectively or individually electronically focusable onto a desired target area. The electronic focusing of ultrasound energy is known in the art.

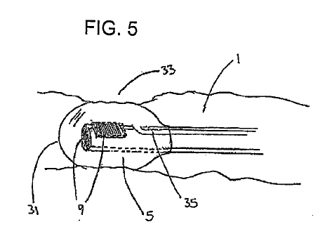

As depicted in FIG. 5, some other preferred embodiments of the ultrasound medical device also include at least one expandable balloon portion 31 disposed on a section or plurality of sections of the distal portion 5 of the insertion piece. In the illustrated embodiment, a single expandable balloon portion 31 surrounds both of two transducer elements 9, and is expanded to contact the surface of the patient's tissue 33 to provide acoustic coupling between the transducer elements 9 and patient tissue 33. One of the expandable portions 31 is preferably a balloon that may be filled with fluid as provided through a lumen 35 in the insertion piece 1.

Alternatively or additionally, a different expandable portion may be provided for anchoring and/or stabilizing the insertion piece within the patient site of interest, a feature important for rendering stationary the insertion piece relative to the imaging or treatment zone. Such a feature is especially critical within the lung, since normal pulmonary motion otherwise interferes with the realization of a stable target area. An expandable portion used for anchoring the insertion piece within the patient site of interest may be a balloon, and may be actuated via fluid or air pressure. An expandable portion used for stabilizing the insertion piece within the patient site of interest may be one or more stents, struts, tines (fixed or retractable), or screws or grasping means (for active fixation). As used herein, the term “expandable” means inflatable, extendable, enlargeable, or capable of being dilated, raised, lowered, widened, or lengthened.

Anchoring of the insertion piece is especially critical when the site of interest is located within a channel, passage, cavity, etc., having a width that is greater than that of the insertion piece, since it can serve to countermand the effects of the movement of the site of interest (e.g., in the lung) relative to the insertion piece. Stabilization of the insertion piece can be important even where the insertion piece fits snugly within the channel, passage, cavity, etc., and primarily functions to ensure that the transducer assembly of the insertion piece remains in constant contact with the site of interest and that no gaps—which could lead to acoustic impedance—exist between the transducer assembly and the tissue located at the site of interest.

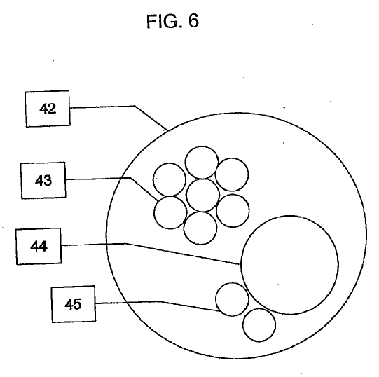

FIG. 6 shows a cross sectional view of one version of the present invention arranged to accommodate a single device 42, housing an optical imaging component 43, an ultrasonic imaging and ablation channel 44 for channeling the connecting wires along the longitudinal axis of the device 42 to the distal tip. With respect to the disclosed methods, any recited imaging step may be performed using the ultrasound transducer(s), the optical imaging component, or both. In addition cooling channels 45 are included to allow the presence of either open or closed loop cooling means flowing from the handle of the insertion piece to the ultrasonic ablation transducer at the distal end. The cooling means can comprise any suitable material capable of being translated over the length of the insertion piece, including solids, semisolids, liquids, or gases, numerous variations of which are readily appreciated by those skilled in the art.

Exemplary devices of the instant invention may be equipped with mechanically or electronically adjustable transducer assemblies or expandable portions or both, each of said features serving to specially adapt the present ultrasound medical devices for use in tissue regions where air spaces exist. All combinations and versions of such adaptations are contemplated as being within the scope of the present invention.

The present ultrasound medical devices provide advantages over traditional medical intervention involving open surgery and/or biopsy. As an alternative to traditional invasive procedures, insertion of the instant ultrasound devices can be easily achieved: in some embodiments, the insertion piece further comprises at least one lumen that is compatible for use with a guide wire, over which the insertion piece may be translated for uncomplicated insertion into a patient. In other embodiments, the device is adapted for endoscopic, transbronchial insertion, for example, through the working channel of a flexible bronchoscope, through a rigid bronchoscope, or other suitable introducer mechanism. Such an adaptation may include the sizing of the insertion piece for compatibility with a bronchoscope and the construction of the insertion piece from materials that afford the necessary rigidity for such use, as provided above. In still other embodiments, the ultrasound device is adapted for percutaneous insertion into a patient; skilled artisans will no doubt be familiar with the sizing and material specifications required for percutaneous insertion. The device may also be adapted for use during open surgery.

Optimally, the instant ultrasound devices are capable of being manually operated. Therefore, the ultrasound medical device may further comprise a hand-piece operatively connected to the insertion piece. The ultrasound imaging and medical treatment functionalities of the instant device are preferably controlled via an ultrasound controlling unit that is operatively connected to the insertion piece. Preferred ultrasound controlling units may be operated by a human or automatically according to preset specifications.

Also provided herein are methods for the imaging, characterization, diagnosis, and/or medical treatment of patient tissue. A highly preferred method involves a) introducing into a patient at a location having or suspected of having an air/tissue interface an insertion piece having a longitudinal axis and a distal end, wherein the insertion piece has a transducer assembly disposed at its distal end, and the transducer assembly comprises one or more transducer elements, each having an ultrasound emitting surface; b) positioning the distal end of said insertion piece at, in, or near a target area; c) activating the device so that at least part of the transducer assembly thereof begins emitting ultrasound, and, d) using said ultrasound emissions for imaging or medical treatment, or for any combination thereof.

The transducer assembly that is on the insertion piece used in the introducing step may be adjustably mounted to provide a source of ultrasound that may be adaptively focused onto a desired target area. Otherwise, the transducer assembly of the insertion piece may, instead of being adjustably mounted, be electronically focusable onto a desired target area.

In contrast with similar methods known in the art, the site of interest with respect to the instant methods may be in or near the oral cavity or pharynx, nasal cavity or pharynx, esophagus, trachea, bronchi, lungs, stomach, or colon. The insertion piece may be introduced into a patient endoscopically, transbronchially, percutaneously, or may be used in an open surgical procedure. As provided above, transbronchial introduction may be achieved through the working channel of a flexible bronchoscope, through a working bronchoscope, or other suitable introducer mechanism. The insertion piece may further comprises at least one lumen that is compatible for use with a guide wire, and in such instances the insertion piece may be introduced into the patient by translation over a guide wire.

The inventive methods may include the additional step of adjusting the transducer assembly to focus the ultrasound emissions therefrom onto the desired target area. In the event that the transducer assembly is, instead of being adjustably mounted, electronically focusable onto a desired target area, such additional step may instead comprise electronically focusing the transducer assembly to focus the ultrasound emissions onto the desired target area. As used herein throughout the instant disclosure, the term “adjust”, unless otherwise specified, includes adjustment of an adjustably mounted transducer assembly and/or focusing of an electronically focusable transducer assembly, or both. Likewise, the term “focus” as used herein, unless otherwise specified, includes the focusing of an electronically focusable transducer assembly and/or adjustment of an adjustably mounted transducer assembly.

In some preferred methods, the imaging or medical treatment step is repeated one or more times. The repetition of the imaging or medical treatment step permits multiple episodes of imaging or treatment with respect to the same target area, which can both permit detailed, multi-viewed imaging of a target area in a patient (which may, as used herein, include specific features), and enhance the precision of any medical treatment to follow. Where the imaging or medical treatment step is repeated, the methods may further include adjusting or electronically focusing the transducer assembly to focus the ultrasound onto the desired target area prior to one or more of the repetition or repetitions of the imaging or medical treatment step.

Also where the imaging or medical treatment step is repeated, and where the methods further include adjusting or electronically focusing the transducer assembly, the repetition or repetitions of the imaging or medical treatment step may include alternating between imaging and medical treatment. For example, a method of the present invention may comprise an initial round of imaging to assess the features of the target area, followed by focusing or adjusting in response to a discrepancy between the placement of the insertion device and the target area, an additional bout of imaging to confirm the adequacy of the focus or adjustment, then medical treatment based on the imaging results, imaging to assess the results of the medical treatment, a final episode medical treatment, and a final occurrence of imaging to confirm the ultimate disposition of the treatment. This sequence of events and others, whether differently sequentially arranged and/or having more or fewer and different steps, are contemplated as being within the scope of the instant invention.

For example, a generic therapy regimen may comprise one or more episodes of imaging, optionally followed by adjustment of the transducer assembly to focus ultrasound onto a desired target area, optionally followed by one or more episodes of imaging, followed by one or more episodes of medical treatment. Such a therapy regimen may be repeated one or more times. Additionally, one or more repetitions of the therapy regimen or another therapy regimen may involve the selection of a different target area.

The disclosed methods may also involve the simultaneous use of imaging and medical treatment. It is therefore apparent that the provided methods permit the highly precise and purposeful, and optionally recursive, ultrasonic characterization and treatment of patient tissue.

Where the imaging or medical treatment step comprises imaging, such imaging may include diagnosis and/or characterization of the site of interest. Ultrasonic imaging in this manner can provide numerous diagnostic and clinical advantages. Such diagnosis and/or characterization can comprise assessing a target area for its status as benign, malignant, or unknown; it can also or otherwise include determining the size, geometry, vascularity, and/or density of the target area. A determination of the size, geometry, vascularity, and/or density of the target area can in turn be used to guide the adjustment of the focus of the ultrasound emissions in order to align the focus with a particularly desired location on or in the target area, and it can also be used to guide the adjustment of the power of the ultrasound emissions so that the power is optimally compatible with additional imaging and/or medical treatment of the target area. The preceding methods can be enhanced by one or more additional repetitions of imaging or medical treatment or a combination thereof. It should therefore be apparent that, especially with respect to target areas associated with air/tissue interfaces, the present methods represent uniquely versatile and effective ultrasound imaging and treatment applications.

Where the imaging or medical treatment step comprises medical treatment, the medical treatment may be applied to the target area in order to reduce the size of the target area; ablate all or part of the target area; induce necrosis in all or part of the target area; induce apoptosis in all or part of the target area; damage or destroy the vasculature associated with the target area; slow or retard the growth of the target area; or, slow or retard the metastasis of the target area; or any combination thereof. Thus, the disclosed methods enable multifunctional high intensity focused ultrasound treatment in target areas that have heretofore been considered unavailable to this variety of treatment.

Additionally, rather than using the present invention in conjunction with a bronchoscope, the optical imaging portion of the bronchoscope may be included in the present device so that the optical and ultrasonic imaging capabilities are contained within the same device. The optical imaging and ultrasonic portions may be co-registered, to allow direct visualization of the ultrasound treatment area, or they may be separately maneuvered. The optical imaging component may be a normal fiber-optic bundle well known to those skilled in the art or may use spectrally encoded confocal microscopy as described in C. Boudoux, Rapid wave-length swept spectrally encoded confocal microscopy OPTICS LETTERS, Vol 27, No. 6, Mar. 15, 2002, and further defined by the work of Guillermo Tearney et al. at Harvard Medical School (Boston, Mass.). As will also be apparent the combination of a co-registered device containing both an optical and ultrasonic imaging and ablation means may have applications outside of bronchoscopy.

In addition, the treatment step may include the ultrasonic activation of a locally-delivered pharmacologic agent or the sonoporation of a pharmacologic agent into the feature. The pharmacologic agent may be delivered to the site of interest through a lumen in the insertion piece itself or through a different delivery technique, for example, through a catheter not directly associated with the insertion piece. Sonoporation through ultrasound can create transient permeability of cell membranes in the presence of microbubbles, thereby permitting passage of a pharmacologic agent into the cellular constituents of a site of interest and contributing an additional, chemical aspect to the treatment described above. Although ultrasonic activation and sonoporation are themselves known in the art and their execution will be readily understood by the skilled artisan, they have not been used in conjunction with the instant methods and therefore represent novel enhanced therapy of pathogenic or abnormal tissue.

The imaging or medical treatment step may include using any of the many different forms of ultrasound that are known to those skilled in the art, including, for example, A, B M, 2D, Doppler, Pulsed-wave Doppler, Continuous-wave Doppler, Color Doppler, Power Doppler, Directional Power Doppler, or Duplex ultrasound, or any combination thereof. The skilled artisan will appreciate that in certain situations some forms of ultrasound may be preferred over others, and will be able to chose the mode of ultrasound operation that is best suited to the desired application.

The insertion device that is introduced into the patient during the course of the disclosed methods may additionally include at least one expandable portion disposed on a section or plurality of sections of the distal portion of the insertion piece. The at least one expandable portion may surround at least one of said the or more transducer elements, and may be expanded to contact the surface of the patient's tissue to provide acoustic coupling between the transducer element(s) and the patient tissue. The expandable portion may also or alternatively anchor and/or stabilize the insertion piece within the patient. Where the insertion device does include an expandable portion, the methods may comprise the additional step of at least partially expanding at least one of the at least one expandable portion before the imaging and/or medical treatment step. If the insertion piece needs to be repositioned during the course of imaging or treatment, an expandable portion used for anchoring, stabilizing, and/or acoustic coupling may be partially or completely deflated, retracted, or withdrawn (as appropriate) after which time repositioning of the insertion piece take place; the user then has the option of redeploying the expandable portion to achieve anchoring, stabilizing, and/or acoustic coupling once again.

The provided methods may also include delivering an ultrasound contrast agent to the target area. The delivery of an ultrasound contrast agent may serve to improve diagnosis by enhancing, for example, the acoustic brightness of the image surface, and/or improve ultrasonic treatment or other treatment by permitting targeted local delivery of therapy. The particular ultrasound contrast agent may be selected according to the location and characteristics of the target area, or according to other criteria known to those skilled in the art.

### EXAMPLES

The present invention is further defined in the following examples. It should be understood that these examples, while indicating certain embodiments of the invention, are given by way of illustration only, and should not be construed as limiting the appended claims. From the preceding discussion and these examples, one skilled in the art may ascertain the essential characteristics of the instant invention, and without departing from the spirit and scope thereof, can make various changes and modifications of the invention to adapt it to various usages and conditions.

### Example 1

### Ultrasound Ablation Performed on a Test Subject

A 26 kg dog was anesthetized and placed on a ventilator. The chest was opened and the left lung exposed. A high intensity focused ultrasound (HIFU) probe consisting of a 5 element annular array operating with a center frequency of 5.5 megahertz (MHz) was placed in a water standoff. The array had a focal zone of 3.7 cm and a focal spot of 0.5 cm×1.0 cm.

A 7.5 MHz ultrasound imaging probe was positioned in the center of the annular array and used both to aim the HIFU probe and to monitor the delivery of ablation energy. The ventilator was paused at full inspiration to simulate a breath hold, and approximately 60 watts/cm³ of HIFU ablation energy was delivered to the surface of the lung for two seconds. A bright spot was immediately apparent on the ultrasound image indicating the formation of an ablated area in the lung tissue. Interestingly and quite unexpectedly, the strong acoustic impedance mismatch between the lung parenchyma and the ambient environment acted as a mirror or reflector to concentrate the ultrasonic ablation energy within the lung tissue, greatly increasing the efficiency of the tissue heating.

After two seconds a clearly ablated zone of appropriately 5 mm×7 mm was apparent on the ultrasound image, and visual inspection of the lung indicated a dark spot where the energy had entered the lung tissue. Presumably as a result of the unexpected reflection effect of the tissue/air interface, the ultrasound energy was considerably stronger than would be contemplated for clinical use. Interestingly, however, there was no evidence of gross tissue disruption by cavitation or evidence of severe bleeding or pneumothorax, any of which would have proven a clinically undesirable effect of the experimental medical treatment device.

### Example 2

### Additional Ultrasound Ablation to Approximate Clinical Standards

A second ablation lesion was made at another site on the test subject's lung. This lesion was created using approximately 50 w/cm³ of HIFU ablation energy delivered for one second, an amount of energy calculated to approach that which would be much more clinically acceptable.

Again, the tissue-to-air interface acted as a strong reflector, concentrating the ablation energy. After the energy delivery, a clear lesion having dimensions of approximately 5 mm×7 mm was visible on the ultrasound image, and visual inspection showed a white area on the surface of the lung where heating had denatured the proteins with resulting cellular death. Histology results showed clearly defined dead zones with well demarcated borders.

The acoustic mismatch between air and tissue prevents the transfer of acoustic energy from one side of the mismatch to the other. Where ultrasound is used within air-filled viscera and is directed against tissue, the interface between the tissue and air causes the reflection of large amounts of ultrasonic energy back into the tissue, thereby producing the surprising result of making the system of introduction of ultrasound energy to tissue greatly more efficient.

With respect to the disclosed examples, the unexpectedly strong reflection and concentration of energy from the tissue/air interface has implications for device design and procedure efficacy. The concentration of ultrasound energy through reflection leads to greater ablating power at the target area. This concentration of energy by strong reflection may be protective of the ultrasound transducer array, and may in turn allow the use of thinner piezoelectric crystals, and therefore smaller ultrasound devices. The strong reflection will also prevent the transmission of energy across the air-filled area of the target zone and minimize the potential for damage to tissue other than in the specific target area This may allow the use of higher frequencies of ultrasound energy, which would as a result permit the use of smaller crystals, and therefore help to reduce overall ablation device dimensions.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. An ultrasound medical device suitable for use in a target area in or near which an air/tissue interface exists comprising:
an insertion piece having a longitudinal axis and a distal end insertable into a patient; and,
a transducer assembly disposed at the distal end of said insertion piece comprising one or more transducer elements each having an ultrasound emitting surface, said transducer assembly being adjustably mounted to provide a source of ultrasound that may be adaptively focused onto a desired target area.

2. The ultrasound medical device of claim 1 wherein the target area is in or near the oral cavity or pharynx, nasal cavity or pharynx, esophagus, trachea, bronchi, lungs, stomach, or colon.

3. The ultrasound medical device of claim 1 wherein said insertion piece has a length of about 20 to about 600 cm and has a diameter of about 1 mm to about 30 mm.

4. The ultrasound medical device of claim 3 wherein said insertion piece is a catheter and has a diameter of about 5 mm or smaller.

5. The ultrasound medical device of claim 3 wherein said insertion piece is adapted for insertion into a patient through a bronchoscope.

6. The ultrasound medical device of claim 3 wherein said insertion piece is adapted for insertion through the interstitial spaces at the surface of a lung.

7. The ultrasound medical device of claim 1 wherein at least one of said one or more transducer elements is disposed at the terminus of the distal end of said insertion piece.

8. The ultrasound medical device of claim 1 wherein at least one of said transducer elements is disposed on the side of the distal end of said insertion piece.

9. The ultrasound medical device of claim 8 wherein the at least one of transducer elements is also disposed at the terminus of the distal end of said insertion piece.

10. The ultrasound medical device of claim 1 wherein at least one of said transducer elements is an ultrasound imaging transducer, and said ultrasound imaging transducer is capable of A, B M, 2D, Doppler, Pulsed-wave Doppler, Continuous-wave Doppler, Color Doppler, Power Doppler, Directional Power Doppler, or Duplex ultrasound imaging, or any combination thereof.

11. The ultrasound medical device of claim 1 wherein at least one of said one or more transducer elements is an ultrasound medical treatment transducer.

12. The ultrasound medical device of claim 1 wherein at least one of said transducer elements in an ultrasound imaging transducer and an ultrasound medical treatment transducer.

13. The ultrasound medical device of claim 1 wherein the ultrasound emitting surface of at least one of said one or more transducers is oriented so that a cross-section thereof is at an angle that is substantially perpendicular to said longitudinal axis of the insertion piece.

14. The ultrasound medical device of claim 1 wherein said transducer assembly includes a plurality N of said transducers and wherein the ultrasound emitting surface of each transducer is oriented at an angle of about 360/N degrees apart from the ultrasound emitting surface of an adjacent transducer.

15. The ultrasound medical device of claim 1 wherein at least one of the one or more transducers features an ultrasound emitting surface the cross-section of which is substantially linear.

16. The ultrasound medical device of claim 1 wherein at least one of the one or more transducers features an ultrasound emitting surface the cross-section of which is substantially concave.

17. The ultrasound medical device of claim 1 further comprising at least one expandable portion disposed on a section or plurality of sections of the distal portion of said insertion piece, wherein said at least one expandable portion may be expanded to contact the surface of said patient's tissue to provide anchoring of said insertion piece, stabilization of said insertion piece, or acoustic coupling between said at least one of said one or more transducer elements and said patient tissue.

18. The ultrasound medical device of claim 17 wherein said at least one expandable portion comprises at least one balloon, stent, strut, tine (fixed or retractable), or screw or grasping means for active fixation.

19. The ultrasound medical device of claim 1 wherein said transducer assembly is adjustably mounted on a tractable surface having an adaptively variable surface profile that may be actuated electronically, hydraulically, pneumatically, magnetically, chemically, through a shape memory metal, mechanically, or any combination thereof.

20. The ultrasound medical device of claim 19 wherein said adaptively variable surface profile varies according to its radius or radii of curvature.

21. The ultrasound medical device of claim 19 wherein said tractable surface is a flexible material.

22. The ultrasound medical device of claim 21 wherein said flexible material comprises a magnetic material that may be deflected magnetically.

23. The ultrasound medical device of claim 21 wherein said flexible material is deflected by mechanical expansion, compression, or a combination thereof.

24. The ultrasound medical device of claim 21 wherein said flexible material is in communication with an interior portion of said insertion piece.

25. The ultrasound medical device of claim 24 wherein said flexible material is deflected by a variable vacuum created through the induction of negative pneumatic or hydraulic pressure within said insertion piece, or is deflected through the induction of positive pneumatic or hydraulic pressure within said insertion piece.

26. The ultrasound medical device of claim 21 wherein said tractable surface comprises a plurality of piezoelectric elements.

27. The ultrasound medical device of claim 21 wherein said tractable surface comprises a plurality of plates that can be adjusted by one or more cables.

28. The ultrasound medical device of claim 1 wherein said insertion piece further comprises at least one lumen that is compatible for use with a guide wire.

29. The ultrasound medical device of claim 1 wherein said device is adapted for introduction into a patient endoscopically, transbronchially, percutaneously, or during open surgery.

30. The ultrasound medical device of claim 1 further comprising a hand-piece operatively connected to the insertion piece.

31. The ultrasound medical device of claim 1 further comprising an ultrasound controlling unit operatively connected to the insertion piece.

32. The ultrasound medical device of claim 1 wherein said insertion piece further comprises an imaging component.

33. The ultrasound medical device of claim 1 wherein said insertion piece further comprises one or more cooling channels.

34. A method of imaging, characterization, diagnosis, and/or medical treatment of patient tissue comprising the steps of:
introducing in a patient at a location having or suspected of having an air/tissue interface, an insertion piece having a longitudinal axis and a distal end;
said insertion piece having a transducer assembly disposed at its distal end, the assembly comprising one or more transducer elements, each having an ultrasound emitting surface, said transducer assembly being adjustably mounted to provide a source of ultrasound that may be adaptively focused onto a desired target area; and,
positioning the distal end of said insertion piece at, in, or near a target area;
activating the device so that at least part of the transducer assembly thereof begins emitting ultrasound, and,
using said ultrasound emissions for imaging or medical treatment, or for any combination thereof.

35. The method of claim 34 wherein said insertion piece further comprises an imaging component, and wherein said imaging component is used for imaging.

36. The method of claim 34 wherein said site of interest is in or near the oral cavity or pharynx, nasal cavity or pharynx, esophagus, trachea, bronchi, lungs, stomach, or colon.

37. The method of claim 34 wherein said insertion piece is introduced into a patient endoscopically, transbronchially, percutaneously, or during open surgery.

38. The method of claim 37 wherein said insertion piece further comprises at least one lumen that is compatible for use with a guide wire.

39. The method of claim 38 wherein said insertion piece is introduced into a patient by translation over a guide wire.

40. The method of claim 34 further comprising adjusting said transducer assembly to focus said ultrasound onto said desired target area.

41. The method of claim 34 wherein said imaging or medical treatment step is repeated one or more times.

42. The method of claim 41 further comprising adjusting said transducer assembly to focus said ultrasound onto said desired target area prior to one or more of said repetition or repetitions of said imaging or medical treatment step.

43. The method of claim 42 wherein said repetition or repetitions of said imaging or medical treatment step includes alternating between imaging and medical treatment.

44. The method of claim 43 wherein said alternation between imaging and medical treatment comprises a therapy regimen comprising one or more episodes of imaging, optionally followed by adjustment of said transducer assembly to focus said ultrasound onto a desired target area, optionally followed by one or more episodes of imaging, followed by one or more episodes of medical treatment.

45. The method of claim 44 wherein said therapy regimen is repeated one or more times.

46. The method of claim 45 wherein one or more of said repetitions of said therapy regimen involves selecting a different target area.

47. The method of claim 34 wherein imaging and medical treatment is performed simultaneously.

48. The method of claim 34 wherein said imaging or medical treatment step comprises imaging, and said imaging comprises diagnosis and/or characterization of said target area.

49. The method of claim 48 wherein said imaging comprises assessing said target area for its status as benign, malignant, or unknown.

50. The method of claim 48 wherein said imaging comprises determining the size, geometry, vascularity, and/or density of said target area.

51. The method of claim 50 wherein said determination of size, geometry, vascularity, and/or density of said target area is used to guide the adjustment of the focus of said ultrasound emissions to align said focus with a desired location on or in said target area.

52. The method of claim 50 wherein said determination of size, geometry, vascularity, and/or density of said target area is used to guide the adjustment of the power of said ultrasound emissions so that said power is optimally compatible with additional imaging and/or medical treatment of said target area.

53. The method of claim 52 further comprising one or more additional repetitions of imaging or medical treatment or a combination thereof.

54. The method of claim 34 wherein said imaging or medical treatment step comprises medical treatment, and said medical treatment is applied to said target area in order to:
reduce the size of said target area; ablate all or part of said target area; induce necrosis in all or part of said target area; induce apoptosis in all or part of said target area; damage or destroy the vasculature associated with said target area; slow or retard the growth of said target area; or, slow or retard the metastasis of said target area,
or any combination thereof.

55. The method of claim 34 wherein said imaging or medical treatment step comprises using A, B M, 2D, Doppler, Pulsed-wave Doppler, Continuous-wave Doppler, Color Doppler, Power Doppler, Directional Power Doppler, or Duplex ultrasound, or any combination thereof.

56. The method of claim 34 wherein said insertion piece further comprises at least one expandable portion disposed on a section or plurality of sections of the distal portion of said insertion piece, wherein said at least one expandable portion may be expanded to contact the surface of said patient's tissue to provide anchoring of said insertion piece, stabilization of said insertion piece, or acoustic coupling between said at least one of said one or more transducer elements and said patient tissue.

57. The method of claim 56 comprising at least partially expanding at least one of said at least one expandable portion before said imaging or medical treatment step.

58. The method of claim 34 further comprising delivering an ultrasound contrast agent to the target area.

59. The method of claim 34 further comprising ultrasonic activation of a locally-delivered pharmacologic agent or the sonoporation of a pharmacologic agent into the target area.

60. A method of imaging, characterization, diagnosis, and/or medical treatment of patient tissue comprising the steps of:
introducing in a patient at a location having or suspected of having an air/tissue interface, an insertion piece having a longitudinal axis and a distal end;
said insertion piece having:
a transducer assembly disposed at its distal end, the assembly comprising one or more transducer elements, each having an ultrasound emitting surface, said transducer assembly being electronically focusable onto a desired target area;
positioning the distal end of said insertion piece at, in, or near a target area;
activating the device so that at least part of the transducer assembly thereof begins emitting ultrasound, and,
using said ultrasound emissions for imaging or medical treatment, or for any combination thereof.

61. The method of claim 60 wherein said insertion piece further comprises an imaging component, and wherein said imaging component is used for imaging.

62. The method of claim 60 wherein said site of interest is in or near the oral cavity or pharynx, nasal cavity or pharynx, esophagus, trachea, bronchi, lungs, stomach, or colon.

63. The method of claim 60 wherein said insertion piece is introduced into a patient endoscopically, transbronchially, percutaneously, or during open surgery.

64. The method of claim 60 wherein said insertion piece further comprises at least one lumen that is compatible for use with a guide wire.

65. The method of claim 64 wherein said insertion piece is introduced into a patient by translation over a guide wire.

66. The method of claim 60 further comprising adjusting said transducer assembly to focus said ultrasound onto said desired target area.

67. The method of claim 60 wherein said imaging or medical treatment step is repeated one or more times.

68. The method of claim 67 further comprising adjusting said transducer assembly to focus said ultrasound onto said desired target area prior to one or more of said repetition or repetitions of said imaging or medical treatment step.

69. The method of claim 68 wherein said repetition or repetitions of said imaging or medical treatment step includes alternating between imaging and medical treatment.

70. The method of claim 69 wherein said alternation between imaging and medical treatment comprises a therapy regimen comprising one or more episodes of imaging, optionally followed by adjustment of said transducer assembly to focus said ultrasound onto a desired target area, optionally followed by one or more episodes of imaging, followed by one or more episodes of medical treatment.

71. The method of claim 70 wherein said therapy regimen is repeated one or more times.

72. The method of claim 71 wherein one or more of said repetitions of said therapy regimen involves selecting a different target area.

73. The method of claim 60 wherein imaging and medical treatment is performed simultaneously.

74. The method of claim 60 wherein said imaging or medical treatment step comprises imaging, and said imaging comprises diagnosis and/or characterization of said target area.

75. The method of claim 74 wherein said imaging comprises assessing said target area for its status as benign, malignant, or unknown.

76. The method of claim 74 wherein said imaging comprises determining the size, geometry, vascularity, and/or density of said target area.

77. The method of claim 76 wherein said determination of size, geometry, vascularity, and/or density of said target area is used to guide the adjustment of the focus of said ultrasound emissions to align said focus with a desired location on or in said target area.

78. The method of claim 76 wherein said determination of size, geometry, vascularity, and/or density of said target area is used to guide the adjustment of the power of said ultrasound emissions so that said power is optimally compatible with additional imaging and/or medical treatment of said target area.

79. The method of claim 78 further comprising one or more additional repetitions of imaging or medical treatment or a combination thereof.

80. The method of claim 60 wherein said imaging or medical treatment step comprises medical treatment, and said medical treatment is applied to said target area in order to:
reduce the size of said target area; ablate all or part of said target area; induce necrosis in all or part of said target area; induce apoptosis in all or part of said target area; damage or destroy the vasculature associated with said target area; slow or retard the growth of said target area; or, slow or retard the metastasis of said target area,
or any combination thereof.

81. The method of claim 60 wherein said imaging or medical treatment step comprises using A, B M, 2D, Doppler, Pulsed-wave Doppler, Continuous-wave Doppler, Color Doppler, Power Doppler, Directional Power Doppler, or Duplex ultrasound, or any combination thereof.

82. The method of claim 60 wherein said insertion piece further comprises at least one expandable portion disposed on a section or plurality of sections of the distal portion of said insertion piece, wherein said at least one expandable portion may be expanded to contact the surface of said patient's tissue to provide anchoring of said insertion piece, stabilization of said insertion piece, or acoustic coupling between said at least one of said one or more transducer elements and said patient tissue.

83. The method of claim 78 comprising at least partially expanding at least one of said at least one expandable portion before said imaging or medical treatment step.

84. The method of claim 60 further comprising delivering an ultrasound contrast agent to the target area.

85. The method of claim 60 further comprising ultrasonic activation of a locally-delivered pharmacologic agent or the sonoporation of a pharmacologic agent into the target area.

86. A method of imaging, characterization, diagnosis, and/or medical treatment of patient tissue comprising the steps of:
introducing in a patient at a location having or suspected of having an air/tissue interface, an insertion piece having a longitudinal axis and a distal end;
said insertion piece having:
a transducer assembly disposed at its distal end, the assembly comprising one or more transducer elements, each having an ultrasound emitting surface; and,
at least one expandable portion disposed on a section or plurality of sections of the distal portion of said insertion piece, wherein said at least one expandable portion may be expanded to contact the surface of said patient's tissue to provide anchoring of said insertion piece, stabilization of said insertion piece, and/or acoustic coupling between said at least one of said one or more transducer elements and said patient tissue;
positioning the distal end of said insertion piece at, in, or near a target area;
activating the device so that at least part of the transducer assembly thereof begins emitting ultrasound, and,
using said ultrasound emissions for imaging or medical treatment, or for any combination thereof.

87. The method according to claim 86, wherein said insertion piece further comprises an imaging component, and wherein said imaging component is used for imaging.

88. The method of claim 86 wherein said site of interest is in or near the oral cavity or pharynx, nasal cavity or pharynx, esophagus, trachea, bronchi, lungs, stomach, or colon.

89. The method of claim 86 wherein said insertion piece is introduced into a patient endoscopically, transbronchially, percutaneously, or during open surgery.

90. The method of claim 86 wherein said insertion piece further comprises at least one lumen that is compatible for use with a guide wire.

91. The method of claim 90 wherein said insertion piece is introduced into a patient by translation over a guide wire.

92. The method of claim 86 wherein said transducer assembly is adjustable and/or focusable to provide a source of ultrasound that may be adaptively focused onto said target area.

93. The method of claim 92 further comprising adjusting said transducer assembly to focus said ultrasound onto said desired target area.

94. The method of claim 93 wherein said imaging or medical treatment step is repeated one or more times.

95. The method of claim 94 further comprising adjusting said transducer assembly to focus said ultrasound onto said desired target area prior to one or more of said repetition or repetitions of said imaging or medical treatment step.

96. The method of claim 95 wherein said repetition or repetitions of said imaging or medical treatment step includes alternating between imaging and medical treatment.

97. The method of claim 96 wherein said alternation between imaging and medical treatment comprises a therapy regimen comprising one or more episodes of imaging, optionally followed by adjustment of said transducer assembly to focus said ultrasound onto a desired target area, optionally followed by one or more episodes of imaging, followed by one or more episodes of medical treatment.

98. The method of claim 97 wherein said therapy regimen is repeated one or more times.

99. The method of claim 98 wherein one or more of said repetitions of said therapy regimen involves selecting a different target area.

100. The method of claim 86 wherein imaging and medical treatment is performed simultaneously.

101. The method of claim 86 wherein said imaging or medical treatment step comprises imaging, and said imaging comprises diagnosis and/or characterization of said target area.

102. The method of claim 101 wherein said imaging comprises assessing said target area for its status as benign, malignant, or unknown.

103. The method of claim 101 wherein said imaging comprises determining the size, geometry, vascularity, and/or density of said target area.

104. The method of claim 103 wherein said determination of size, geometry, vascularity, and/or density of said target area is used to guide the adjustment of the focus of said ultrasound emissions to align said focus with a desired location on or in said target area.

105. The method of claim 101 wherein said determination of size, geometry, vascularity, and/or density of said target area is used to guide the adjustment of the power of said ultrasound emissions so that said power is optimally compatible with additional imaging and/or medical treatment of said target area.

106. The method of claim 105 further comprising one or more additional repetitions of imaging or medical treatment or a combination thereof.

107. The method of claim 86 wherein said imaging or medical treatment step comprises medical treatment, and said medical treatment is applied to said target area in order to:
reduce the size of said target area; ablate all or part of said target area; induce necrosis in all or part of said target area; induce apoptosis in all or part of said target area; damage or destroy the vasculature associated with said target area; slow or retard the growth of said target area; or, slow or retard the metastasis of said target area,
or any combination thereof.

108. The method of claim 86 wherein said imaging or medical treatment step comprises using A, B M, 2D, Doppler, Pulsed-wave Doppler, Continuous-wave Doppler, Color Doppler, Power Doppler, Directional Power Doppler, or Duplex ultrasound, or any combination thereof.

109. The method of claim 86 further comprising at least partially expanding at least one of said at least one expandable portion before said imaging or medical treatment step.

110. The method of claim 109 wherein said at least one expandable portion comprises at least one balloon, stent, strut, tine (fixed or retractable), or screw or grasping means for active fixation, and may be expanded to contact the surface of said patient's tissue to provide anchoring of said insertion piece, stabilization of said insertion piece, or acoustic coupling between said at least one of said one or more transducer elements and said patient tissue.

111. The method of claim 86 further comprising delivering an ultrasound contrast agent to the target area.

112. The method of claim 86 further comprising ultrasonic activation of a locally-delivered pharmacologic agent or the sonoporation of a pharmacologic agent into the target area.

113. An ultrasound medical device suitable for use in a target area in or near which an air/tissue interface exists comprising:
an insertion piece having a longitudinal axis and a distal end insertable into a patient;
a transducer assembly disposed at the distal end of said insertion piece comprising one or more transducer elements each having an ultrasound emitting surface; and,
at least one expandable portion disposed on a section or plurality of sections of the distal portion of said insertion piece, wherein said at least one expandable portion may be expanded to contact the surface of said patient's tissue to provide anchoring of said insertion piece, stabilization of said insertion piece, and/or acoustic coupling between said at least one of said one or more transducer elements and said patient tissue.

114. The ultrasound medical device of claim 113 further comprising an imaging component disposed at the distal end of said insertion piece.

115. The ultrasound medical device of claim 113 wherein said insertion piece further comprises one or more cooling channels.

116. The ultrasound medical device of claim 113 wherein the target area is in or near the oral cavity or pharynx, nasal cavity or pharynx, esophagus, trachea, bronchi, lungs, stomach, or colon.

117. The ultrasound medical device of claim 113 wherein said insertion piece has a length of about 20 to about 600 cm and has a diameter of about 1 mm to about 30 mm.

118. The ultrasound medical device of claim 117 wherein said insertion piece is a catheter and has a diameter of about 5 mm or smaller.

119. The ultrasound medical device of claim 117 wherein said insertion piece is adapted for insertion into a patient through a bronchoscope.

120. The ultrasound medical device of claim 117 wherein said insertion piece is adapted for insertion through the interstitial spaces at the surface of a lung.

121. The ultrasound medical device of claim 113 wherein at least one of said one or more transducer elements is disposed at the terminus of the distal end of said insertion piece.

122. The ultrasound medical device of claim 113 wherein at least one of said transducer elements is disposed on the side of the distal end of said insertion piece.

123. The ultrasound medical device of claim 122 wherein the at least one of transducer elements is also disposed at the terminus of the distal end of said insertion piece.

124. The ultrasound medical device of claim 113 wherein at least one of said transducer elements is an ultrasound imaging transducer, and said ultrasound imaging transducer is capable of A, B M, 2D, Doppler, Pulsed-wave Doppler, Continuous-wave Doppler, Color Doppler, Power Doppler, Directional Power Doppler, or Duplex ultrasound imaging, or any combination thereof.

125. The ultrasound medical device of claim 113 wherein at least one of said one or more transducer elements is an ultrasound medical treatment transducer.

126. The ultrasound medical device of claim 113 wherein at least one of said transducer elements in an ultrasound imaging transducer and an ultrasound medical treatment transducer.

127. The ultrasound medical device of claim 113 wherein the ultrasound emitting surface of at least one of said one or more transducers is oriented so that a cross-section thereof is at an angle that is substantially perpendicular to said longitudinal axis of the catheter.

128. The ultrasound medical device of claim 113 wherein said transducer assembly includes a plurality N of said transducers and wherein the ultrasound emitting surface of each transducer is oriented at an angle of about 360/N degrees apart from the ultrasound emitting surface of an adjacent transducer.

129. The ultrasound medical device of claim 113 wherein at least one of the one or more transducers features an ultrasound emitting surface the cross-section of which is substantially linear.

130. The ultrasound medical device of claim 113 wherein at least one of the one or more transducers features an ultrasound emitting surface the cross-section of which is substantially concave.

131. The ultrasound medical device of claim 113 wherein said at least one expandable portion comprises at least one balloon, stent, strut, tine (fixed or retractable), or screw or grasping means for active fixation.

132. The ultrasound medical device of claim 113 wherein said transducer assembly is adjustably mounted to provide a source of ultrasound that may be adaptively focused onto a desired target area.

133. The ultrasound medical device of claim 132 wherein said transducer assembly is adjustably mounted on a tractable surface having an adaptively variable surface profile that may be actuated electronically, hydraulically, pneumatically, magnetically, chemically, through a shape memory metal, or mechanically.

134. The ultrasound medical device of claim 132 wherein said adaptively variable surface profile varies according to its radius or radii of curvature.

135. The ultrasound medical device of claim 133 wherein said tractable surface is a flexible material.

136. The ultrasound medical device of claim 135 wherein said flexible material comprises a magnetic material that may be deflected magnetically.

137. The ultrasound medical device of claim 135 wherein said flexible material is deflected by mechanical expansion, compression, or a combination thereof.

138. The ultrasound medical device of claim 135 wherein said flexible material is in communication with an interior portion of said insertion piece.

139. The ultrasound medical device of claim 138 wherein said flexible material is deflected by a variable vacuum created through the induction of negative pneumatic or hydraulic pressure within said insertion piece, or is deflected through the induction of positive pneumatic or hydraulic pressure within said insertion piece.

140. The ultrasound medical device of claim 133 wherein said tractable surface comprises a plurality of piezoelectric elements.

141. The ultrasound medical device of claim 133 wherein said tractable surface comprises a plurality of plates that can be adjusted by one or more cables.

142. The ultrasound medical device of claim 113 wherein said insertion piece further comprises at least one lumen that is compatible for use with a guide wire.

143. The ultrasound medical device of claim 113 wherein said device is adapted for introduction into a patient endoscopically, transbronchially, percutaneously, or during open surgery.

144. The ultrasound medical device of claim 113 further comprising a hand-piece operatively connected to the insertion piece.

145. The ultrasound medical device of claim 113 further comprising an ultrasound controlling unit operatively connected to the insertion piece.

146. The ultrasound medical device of claim 113 wherein said transducer assembly is electronically focusable onto a desired target area.

147. An ultrasound medical device suitable for use in a target area in or near which an air/tissue interface exists comprising:
an insertion piece having a longitudinal axis and a distal end insertable into a patient; and,
a transducer assembly disposed at the distal end of said insertion piece comprising one or more transducer elements each having an ultrasound emitting surface, said transducer assembly being electronically focusable onto a desired target area.

148. The ultrasound medical device of claim 147 further comprising an imaging component disposed at the distal end of said insertion piece.

149. The ultrasound medical device of claim 147 wherein said insertion piece further comprises one or more cooling channels.

150. The ultrasound medical device of claim 147 wherein the target area is in or near the oral cavity or pharynx, nasal cavity or pharynx, esophagus, trachea, bronchi, lungs, stomach, or colon.

151. The ultrasound medical device of claim 147 wherein said insertion piece has a length of about 20 to about 600 cm and has a diameter of about 1 mm to about 30 mm.

152. The ultrasound medical device of claim 151 wherein said insertion piece is a catheter and has a diameter of about 5 mm or smaller.

153. The ultrasound medical device of claim 151 wherein said insertion piece is adapted for insertion into a patient through a bronchoscope.

154. The ultrasound medical device of claim 151 wherein said insertion piece is adapted for insertion through the interstitial spaces at the surface of a lung.

155. The ultrasound medical device of claim 147 wherein at least one of said one or more transducer elements is disposed at the terminus of the distal end of said insertion piece.

156. The ultrasound medical device of claim 147 wherein at least one of said transducer elements is disposed on the side of the distal end of said insertion piece.

157. The ultrasound medical device of claim 156 wherein the at least one of transducer elements is also disposed at the terminus of the distal end of said insertion piece.

158. The ultrasound medical device of claim 147 wherein at least one of said transducer elements is an ultrasound imaging transducer, and said ultrasound imaging transducer is capable of A, B M, 2D, Doppler, Pulsed-wave Doppler, Continuous-wave Doppler, Color Doppler, Power Doppler, Directional Power Doppler, or Duplex ultrasound imaging, or any combination thereof.

159. The ultrasound medical device of claim 147 wherein at least one of said one or more transducer elements is an ultrasound medical treatment transducer.

160. The ultrasound medical device of claim 147 wherein at least one of said transducer elements in an ultrasound imaging transducer and an ultrasound medical treatment transducer.

161. The ultrasound medical device of claim 147 wherein the ultrasound emitting surface of at least one of said one or more transducers is oriented so that a cross-section thereof is at an angle that is substantially perpendicular to said longitudinal axis of the insertion piece.

162. The ultrasound medical device of claim 147 wherein said transducer assembly includes a plurality N of said transducers and wherein the ultrasound emitting surface of each transducer is oriented at an angle of about 360/N degrees apart from the ultrasound emitting surface of an adjacent transducer.

163. The ultrasound medical device of claim 147 wherein at least one of the one or more transducers features an ultrasound emitting surface the cross-section of which is substantially linear.

164. The ultrasound medical device of claim 147 wherein at least one of the one or more transducers features an ultrasound emitting surface the cross-section of which is substantially concave.

165. The ultrasound medical device of claim 147 further comprising at least one expandable portion disposed on a section or plurality of sections of the distal portion of said insertion piece, wherein said at least one expandable portion may be expanded to contact the surface of said patient's tissue to provide anchoring of said insertion piece, stabilization of said insertion piece, or acoustic coupling between said at least one of said one or more transducer elements and said patient tissue.

166. The ultrasound medical device of claim 165 wherein said at least one expandable portion comprises at least one balloon, stent, strut, tine (fixed or retractable), or screw or grasping means for active fixation.

167. The ultrasound medical device of claim 147 wherein said transducer assembly is at least partly adjustably mounted on a tractable surface having an adaptively variable surface profile that may be actuated electronically, hydraulically, pneumatically, magnetically, chemically, through a shape memory metal, mechanically, or any combination thereof to provide a source of ultrasound that may be adaptively focused onto a desired target area.

168. The ultrasound medical device of claim 167 wherein said adaptively variable surface profile varies according to its radius or radii of curvature.

169. The ultrasound medical device of claim 159 wherein said tractable surface is a flexible material.

170. The ultrasound medical device of claim 169 wherein said flexible material comprises a magnetic material that may be deflected magnetically.

171. The ultrasound medical device of claim 169 wherein said flexible material is deflected by mechanical expansion, compression, or a combination thereof.

172. The ultrasound medical device of claim 169 wherein said flexible material is in communication with an interior portion of said insertion piece.

173. The ultrasound medical device of claim 172 wherein said flexible material is deflected by a variable vacuum created through the induction of negative pneumatic or hydraulic pressure within said insertion piece, or is deflected through the induction of positive pneumatic or hydraulic pressure within said insertion piece.

174. The ultrasound medical device of claim 169 wherein said tractable surface comprises a plurality of piezoelectric elements.

175. The ultrasound medical device of claim 169 wherein said tractable surface comprises a plurality of plates that can be adjusted by one or more cables.

176. The ultrasound medical device of claim 147 wherein said insertion piece further comprises at least one lumen that is compatible for use with a guide wire.

177. The ultrasound medical device of claim 147 wherein said device is adapted for introduction into a patient endoscopically, transbronchially, percutaneously, or during open surgery.

178. The ultrasound medical device of claim 147 further comprising a hand-piece operatively connected to the insertion piece.

179. The ultrasound medical device of claim 147 further comprising an ultrasound controlling unit operatively connected to the insertion piece.
